(19) United States
(12) Patent Application Publication
(10) Pub. No.: US 2024/0331361 A1
(43) Pub. Date: Oct. 3, 2024

(54) METHOD FOR TRAINING A MEDICAL IMAGE CLASSIFICATION MODEL USING MULTI-FILTER AUTO-AUGMENTATION

(71) Applicant: KNU-Industry Cooperation Foundation, Chuncheon-si (KR)

(72) Inventors: Hyun-chong CHO, Chuncheon-si (KR); Jung-woo CHAE, Chuncheon-si (KR)

(73) Assignee: KNU-Industry Cooperation Foundation, Chuncheon-si (KR)

(21) Appl. No.: 18/622,282

(22) Filed: Mar. 29, 2024

(30) Foreign Application Priority Data
Mar. 30, 2023 (KR) 10-2023-0041881

(51) Int. Cl. G06V 10/774; G06N 3/045; G06V 10/764; G06V 10/82; G16H 30/40

(52) U.S. Cl. CPC G06V 10/774; G06N 3/045; G06V 10/764; G06V 10/82; G16H 30/40; G06V 2201/03

(57) ABSTRACT

A method for training a medical image classification model using a multi-filter auto-augmentation includes training, by using a training dataset including raw medical image data, a plurality of first neural network models to classify medical image data into a predetermined class, in which the plurality of first neural network models have different neural network model structures, auto-augmenting the raw medical image data to generate medical image augmentation data, filtering data of the medical image augmentation data, which has a class probability of belonging to a class classified by each of the plurality of first neural network models, equal to or greater than a predetermined criterion, as effective augmentation data, and training, by using a training dataset including the effective augmentation data and the raw medical image data, a second neural network model to classify medical image data into a predetermined class.

### CROSS-REFERENCE TO RELATED APPLICATION

This application claims priority to Korean Patent Application No. 10-2023-0041881, filed in the Korean Intellectual Property Office on Mar. 30, 2023, the entire contents of which are hereby incorporated by reference.

### BACKGROUND

### Technical Field

The present disclosure relates to a method for training a medical image classification model, and more specifically, to a method for multi-filtering auto-augmented training data and training a neural network model, and classifying a medical image using the trained neural network model.

### Description of the Related Art

Gastroscopy is considered to be the best method for early diagnosis of gastrointestinal cancer and is commonly used in many countries, and technology has been increasingly advanced with the use of high-performance equipment. In addition, this method has the advantage of being able to help medical professionals diagnose early gastrointestinal cancer and precancerous lesions at a reasonable cost without side effects for patients. However, because gastroscopy requires visual observation by an expert, factors such as fatigue and skill of the expert affect the diagnosis result. To solve these problems, computer-assisted diagnosis (CADx) systems can help by analyzing medical data and providing auxiliary expert opinions. In recent years, research on deep learning-based CADx systems has been actively conducted. These systems quickly provide useful information to experts based on accumulated medical data. In addition, by preventing the subjective determination or misdiagnosis of experts, the accuracy of diagnosis can be improved and the burden on experts can be reduced. However, medical data collection, which is the most important step in the development of CADx systems based on deep learning, first requires a prerequisite that lesions will occur, and approval procedures and patient consent to protect patient information are essential. The amount of medical data that can be obtained through this process is inevitably smaller than the amount of other types of data, and this is a disadvantage in deep learning-based CADx systems where the quantity and quality of data are the most important factors for performance.

### SUMMARY

A technical problem to be solved by the present disclosure is to provide a method for multi-filtering auto-augmented training data and training a neural network model, and classifying a medical image using the neural network model trained as a result.

### Solution to Problem

In order to solve the above and other technical problems, a method for training medical image classification model using a computing device according to the present disclosure may include training, by using a training dataset including raw medical image data, a plurality of first neural network models to classify medical image data into a predetermined class, in which the plurality of first neural network models may have different neural network model structures, auto-augmenting the raw medical image data to generate medical image augmentation data, filtering data of the medical image augmentation data, which has a class probability of belonging to a class classified by each of the plurality of first neural network models, equal to or greater than a predetermined criterion, as effective augmentation data, and training, by using a training dataset including the effective augmentation data and the specific raw medical image data, a second neural network model to classify medical image data into a predetermined class.

The effective augmentation data may be obtained by sequentially filtering the medical image augmentation data with the plurality of first neural network models trained with the raw medical image data.

One of the plurality of first neural network models and the second neural network model may have the same neural network model structure.

The plurality of first neural network models and the second neural network models may be deep neural networks (DNNs).

A method for classifying a medical image using the computing device to solve the technical problems according to the present disclosure may include classifying medical image data into the predetermined class by using the second neural network model trained by the method for training the medical image classification model.

An embodiment of the present disclosure for solving the technical problem described above includes a computer-readable recording medium recording a program for executing the method for training the medical image classification model described above.

A computing device for solving the technical problems described above according to an embodiment of the present disclosure may include a processor, and a memory that stores an instruction or program executable by the processor, in which, when the instruction or program is executed by the processor, the method for training the medical image classification model may be executed.

A computing device for solving the technical problems described above according to an embodiment of the present disclosure may include a processor, and a memory that stores an instruction or program executable by the processor, in which, when the instruction or program is executed by the processor, the method for classifying the medical image may be executed.

According to the embodiments of the present disclosure, because a deep learning model can be trained while securing sufficient high-quality training data and also reducing the time and cost required to build the training dataset, it is possible to improve the class probability of the trained deep learning model compared to existing deep learning-based systems.

### BRIEF DESCRIPTION OF THE DRAWINGS

The above and other objects, features and advantages of the present disclosure will become more apparent to those of ordinary skill in the art by describing in detail exemplary examples thereof with reference to the accompanying drawings, in which:

FIG. 1 is a diagram schematically illustrating a configuration of a computing device for use with a method for training a medical image classification model according to an embodiment of the present disclosure;

FIG. 2 is a flowchart illustrating a method for training a medical image classification model according to an embodiment of the present disclosure;

FIG. 3 is a diagram conceptually illustrating a method for training a medical image classification model according to an embodiment of the present disclosure; and

FIG. 4 illustrates an example of multi-filtering medical image augmentation data according to an embodiment of the present disclosure.

### DETAILED DESCRIPTION

Hereinafter, certain embodiments will be described in detail with reference to the accompanying drawings to help those with ordinary knowledge in the art easily achieve the present disclosure.

The terms are used herein for the purpose of describing the embodiments and not intended to limit the present disclosure. In the description, a singular expression also includes a plural expression unless specifically stated otherwise in the context. The terms “comprises” and/or “comprising” as used herein do not foreclose the presence or addition of one or more components other than the specified component. Throughout the description, the same reference numerals refer to the same components, and “and/or” includes each and combinations of one or more of the specified components. The terms “first”, “second”, etc. are used to describe various components, but it goes without saying that these components are not limited by these terms. These terms are only used to distinguish one component from another. Therefore, it goes without saying that a first component mentioned below may be a second component within the technical idea of the present disclosure.

A “computing device” as used herein includes all various devices capable of performing computations and providing results to a user. For example, a computing devices may include desktop PCs, notebook computers, and server computers, as well as smart phones, tablet PCs, cellular phones, PCS phones (Personal Communication Service phones), synchronous and asynchronous IMT-2000 (International Mobile Telecommunication-2000) mobile terminals, palm personal computers (Palm PCs), and personal digital assistants (PDAs).

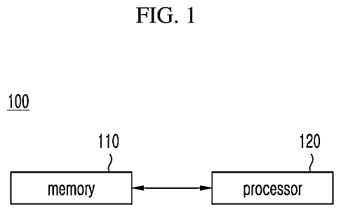

FIG. 1 is a diagram schematically illustrating a configuration of a computing device for use with a method for training a medical image classification model according to an embodiment of the present disclosure.

Referring to FIG. 1, a computing device 100 may include a memory 110 and a processor 120.

The memory 110 may store one or more instructions and/or programs. In addition, the memory 110 may store data used for various tasks related to the method for training a medical image classification model and/or a method for classifying medical image in the computing device 100.

The processor 120 may execute the instructions and/or the computer programs stored in the memory 110 to execute the method for training the medical image classification model and/or the method for classifying the medical image in the computing device 100.

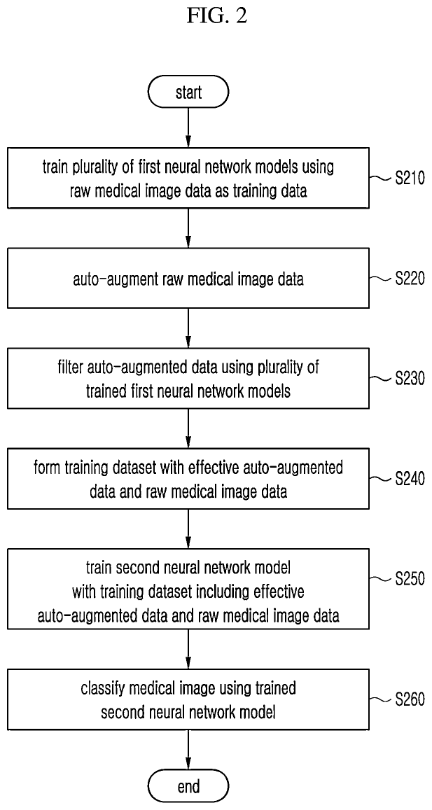

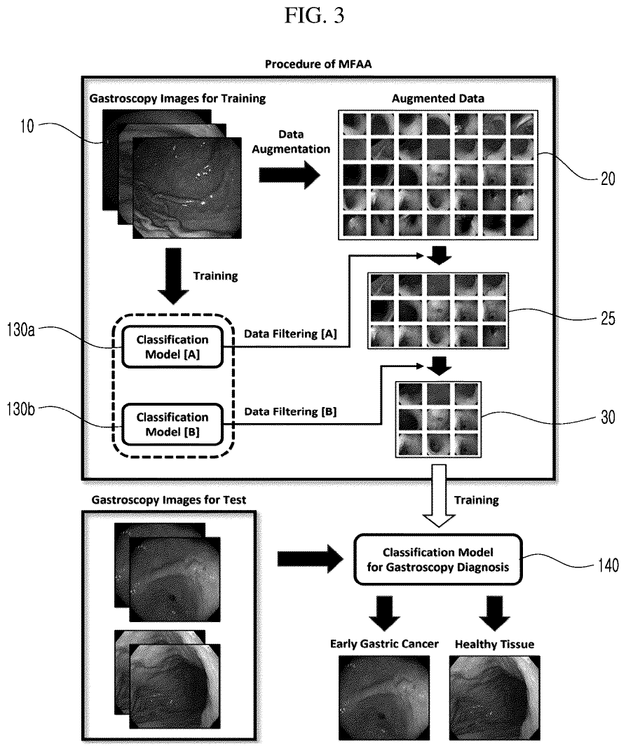

FIG. 2 is a flowchart illustrating the method for training the medical image classification model according to an embodiment of the present disclosure, and FIG. 3 is a diagram conceptually illustrating the method for training the medical image classification model according to an embodiment of the present disclosure.

Referring to FIGS. 2 and 3, first, the computing device 100 may execute the instructions and/or programs on the memory 110 to train a plurality of first neural network models 130a and 130b having different neural network model structures to classify medical image data into predetermined classes (e.g., normal tissue image and gastric lesion image, gastric lesion image and early gastric cancer image, etc.), at S210.

The plurality of first neural network models 130a and 130b may use deep neural network models having different neural network model structures. For example, the first neural network model 130a may use a Vision Transformer (VT) model, and another first neural network model 130b may use a Big Transfer (BiT) model. Of course, according to an embodiment, the first neural network models 130a and 130b may use neural network models other than those described above.

The training dataset used for training the plurality of first neural network models 130a and 130b at S210 uses the raw medical image dataset.

The raw medical image dataset may be raw gastroscopic image dataset 10 as illustrated in FIG. 3. The raw gastroscopic image may be a dataset including gastric lesion images (gastric lesion images including early gastric cancer) and normal tissue images (healthy tissue images without gastric lesions). In this case, the first neural network models 130a and 130b may be trained to receive gastroscopic image data and classify the same into a gastric lesion image and a normal tissue image, at S210.

According to an embodiment, the raw gastroscopic image may be a dataset including an early gastric cancer image and a gastric lesion image (a gastric lesion image other than the early gastric cancer). In this case, the first neural network models 130a and 130b may be trained to receive gastroscopic image data and classify the same into a gastric lesion image and an early gastric cancer image.

Of course, according to an embodiment, the neural network model may also be trained to classify image data into multiple classes such as a normal tissue image, a gastric lesion image, an early gastric cancer image, etc.

Meanwhile, the computing device 100 may auto-augment the raw medical image data to generate auto-augmented medical image data 20 (hereinafter, “medical image augmentation data”), at S220. After setting an augmentation policy(S) and a verification accuracy (R), the computing device 100 may select an efficient augmentation policy for the raw medical image data by adjusting S to increase R through a recursive neural network, and automatically auto-augment the raw medical image data according to the selected augmentation policy. That is, in FIG. 3, the medical image augmentation data 20 represents data auto-augmented from the raw gastroscopic image.

Then, the computing device 100 may filter the medical image augmentation data 20 generated at S220 using the plurality of first neural network models 130a and 130b trained at S210, at S230. Specifically, data of medical image augmentation data, which has a class probability of belonging to the classes classified by each of the plurality of first neural network models 130a and 130b, equal to or greater than a predetermined criterion, may be filtered as effective augmentation data.

When the first neural network models 130a and 130b are trained to classify the medical image into a normal tissue image and a gastric lesion image, if the data classified as the normal tissue image has the normal tissue class probability equal to or greater than a predetermined criterion (e.g., 0.9), the corresponding data may be filtered as the effective data. On the other hand, if the normal tissue class probability of the data classified as the normal tissue image is less than a predetermined criterion (e.g., 0.9), the data may be treated as not effective. Likewise, if a gastric lesion class probability of the data classified as the gastric lesion image is equal to or greater than a predetermined criterion (e.g., 0.9), the data may be filtered as effective, whereas the data may be treated as not effective if the gastric lesion class probability is less than a predetermined criterion (e.g., 0.9).

As illustrated in FIG. 3, the effective augmentation data 30 may be obtained by sequentially filtering the medical image augmentation data 20 using the plurality of first neural network models 130a and 130b. For example, when the filtering criterion is set to a class probability of 0.9, data 25 having a class probability equal to or greater than a predetermined criterion of the medical image augmentation data 20 may be filtered as effective data using the first neural network model 130a (Data Filtering [A] in FIG. 3). In addition, data 30 of the data 25, which has a class probability equal to or greater than a predetermined criterion, may be filtered as effective data using the first neural network model 130b (Data Filtering [B] in FIG. 3).

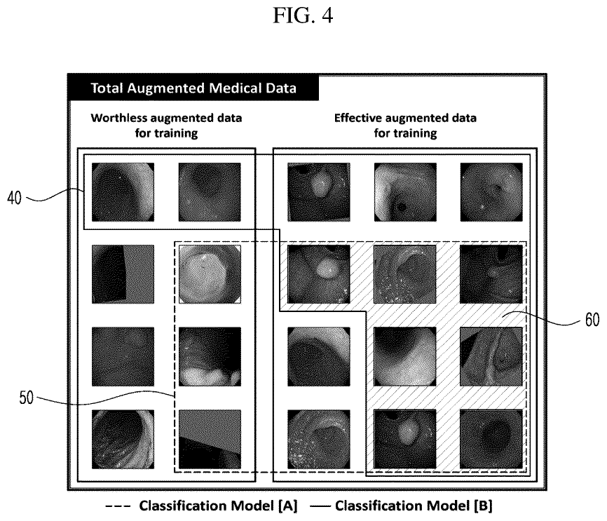

FIG. 4 illustrates an example of multi-filtering medical image augmentation data according to an embodiment of the present disclosure.

Referring to FIG. 4, an area 40 shows that the data is divided into worthless augmented data for training and effective augmented data for training by Classification Model [B]. In addition, an area 50 shows that the data is divided into worthless augmented data for training and effective augmented data for training by Classification Model [A]. According to the method for multi-filter auto-augmenting according to the present disclosure, the data belonging to an area 60, that is, the data filtered as effective by both the Classification Model [A] and the Classification Model [B] may be finally filtered as effective augmentation data.

Referring back to FIG. 2, the computing device 100 may form a training dataset for training a second neural network model 140 with the effective augmentation data 30 filtered at S230 and the raw medical image data 10, at S240. The second neural network model 140 may have the same neural network model structure as one of the plurality of first neural network models 130a and 130b.

Then, the computing device 100 may train the second neural network model 140 to receive medical image data and classify the medical image data into predetermined classes (e.g., normal tissue image and gastric lesion image, gastric lesion image and early gastric cancer image, normal tissue image, gastric lesion image and early gastric cancer image, or the like) using the training dataset formed at S240, at S250.

Finally, the medical image data may be classified into a predetermined class using the second neural network model 140 trained at S250 and output to be used for medical image diagnosis, at S260.

The embodiments described above may be implemented as a hardware component, a software component, and/or a combination of a hardware component and a software component. For example, the devices, methods, and components described in the embodiments may be implemented by using one or more general computing device or specific-purpose computing device such as a processor, a controller, an arithmetic logic unit (ALU), a digital signal processor, a microcomputer, a field programmable gate array (FPGA), a programmable logic unit (PLU), a microprocessor, or any other device capable of executing instructions and responding thereto. The processing device may execute an operating system (OS) and one or more software applications executed on the operating system. Further, the processing device may access, store, operate, process, and generate data in response to the execution of software. For convenience of understanding, it is described in certain examples that one processing device is used, but one of ordinary skill in the art may understand that the processing device may include a plurality of processing elements and/or a plurality of types of processing elements. For example, the processing device may include a plurality of processors or one processor and one controller. In addition, other processing configurations such as a parallel processor are possible.

The software may include a computer program, code, instructions, or a combination of one or more of the above, and may configure the processing unit, or instruct the processing unit independently or collectively to operate as desired. Software and/or data may be interpreted by the processing device or, in order to provide instructions or data to the processing device, may be embodied in any type of machine, component, physical device, virtual equipment, computer storage medium or device, or signal wave transmission, permanently or temporarily. The software may be distributed over networked computer systems and stored or executed in a distributed manner. The software and data may be stored on one or more computer-readable recording media.

The method according to the embodiment may be implemented in the form of program instructions that can be executed through various computer means and recorded in a computer-readable medium. The computer readable medium may include program instructions, data files, data structures, and the like alone or in combination. The program instructions recorded on the medium may be those specially designed and configured for the purposes of the embodiments, or may be known and available to those skilled in computer software. Examples of computer readable recording medium include magnetic media such as hard disks, floppy disks, and magnetic tape, optical media such as CD-ROMs and DVDs, magneto-optical media such as floptical disks, and hardware devices specifically configured to store and execute program instructions such as ROM, RAM, flash memory, and the like. Examples of the program instructions include machine language codes such as those generated by a compiler, as well as high-level language codes that may be executed by a computer using an interpreter, and so on. The hardware device described above may be configured to operate as one or more software modules in order to perform the operations according to the embodiments, and vice versa.

As described above, although the embodiments have been described with reference to the limited drawings, a person of ordinary skill in the art can apply various technical modifications and variations based on the above. For example, even when the described techniques are performed in the order different from the method described above, and/or even when the components of the system, structure, device, circuit, and the like are coupled or combined in a form different from the way described above, or replaced or substituted by other components or equivalents, an appropriate result can be achieved.

1. A method for training a medical image classification model using a computing device, the method comprising:
training, by using a training dataset including raw medical image data, a plurality of first neural network models to classify medical image data into a predetermined class, wherein the plurality of first neural network models have different neural network model structures;
auto-augmenting the raw medical image data to generate medical image augmentation data;
filtering data of the medical image augmentation data, which has a class probability of belonging to a class classified by each of the plurality of first neural network models, equal to or greater than a predetermined criterion, as effective augmentation data; and
training, by using a training dataset including the effective augmentation data and the raw medical image data, a second neural network model to classify medical image data into a predetermined class.

2. The method of claim 1, wherein the effective augmentation data is obtained by sequentially filtering the medical image augmentation data with the plurality of first neural network models trained with the raw medical image data.

3. The method of claim 1, wherein one of the plurality of first neural network models and the second neural network model have a same neural network model structure.

4. The method of claim 1, wherein the plurality of first neural network models and the second neural network models are deep neural networks (DNNs).

5. A method for classifying a medical image, the method comprising classifying medical image data into a predetermined class by using a second neural network model trained by the method for training the medical image classification model according to claim 1.

6. A computer-readable recording medium recording a program for executing the method for training the medical image classification model according to claim 1.
